(19) United States
(12) Patent Application Publication
(10) Pub. No.: US 2026/0146033 A1
(43) Pub. Date: May 28, 2026

(54) Process of Making 2,2,6,6,7,8,8-heptamethyl-3,6,7,8-tetrahydro-2H-indeno[4,5-b]furan and Its Uses

(71) Applicant: INTERNATIONAL FLAVORS & FRAGRANCES INC., NEWYORK, NY (US)

(72) Inventors: Antonio Leyva-Pérez, Valencia (ES); Sergio SANZ-NAVARRO, Valencia (ES); Jorge SÁNCHEZ QUESADA, Benicarlo (ES)

(21) Appl. No.: 19/121,095

(22) PCT Filed: Oct. 9, 2023
(86) PCT No.: PCT/EP2023/077879

§ 371 Date: Apr. 14, 2025

(30) Foreign Application Priority Data
Oct. 21, 2022 (EP) 22383018.3

(51) Int. Cl. C07D 307/93; B01J 23/44; B01J 23/46; B01J 29/08; B01J 29/70; B01J 31/02

(52) U.S. Cl. CPC C07D 307/93; B01J 23/44; B01J 23/462; B01J 23/464; B01J 29/082; B01J 29/084; B01J 29/7007; B01J 31/0225

(57) ABSTRACT

This disclosure relates to a process which involves using 1,1,2,3,3-pentamethyl-5-(2-methylallyl)-1,2,3,5,6,7-hexahydro-4H-inden-4-one (Methallyl Indomuscone) and/or 5-(2-hydroxy-2-methylpropyl)-1, 1,2,3,3-pentamethyl-1,2,3,5,6,7-hexahydro-4H-inden-4-one (Hydroxyisobutyl Indomuscone) as starting material to make 2,2,6,6,7,8,8-heptamethyl-3,6,7,8-tetrahydro-2H-indeno[4,5-b]furan (Aromatic Cyclized Compound). This disclosure also relates to an anaerobic process which involves using Methallyl Indomuscone and/or Hydroxyisobutyl Indomuscone as starting material to make intermediate compound(s) which can be used to make Aromatic Cyclized Compound or 2,2,6,6,7,8,8-heptamethyldecahydro-2H-indeno[4,5-b]furan (Musk Indenofuran). This disclosure also relates to a process for making Musk Indenofuran through Aromatic Cyclized Compound as an intermediate. This disclosure also relates to a one-pot process for making Musk Indenofuran by using Hydroxyisobutyl Indomuscone as the starting material. This disclosure also relates to a phosphine derivative compound of structural formula (X) and a process of making it.

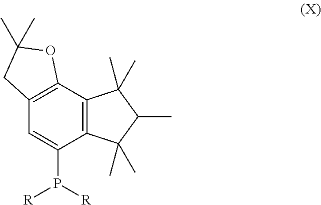

wherein R is selected from the group consisting of alkyl group, cycloalkyl group, phenyl group, substituted phenyl group, and perfluorinated alkyl group.

### BACKGROUND

### Field of the Disclosure

The present disclosure relates to a catalytic process of making 2,2,6,6,7,8,8-heptamethyl-3,6,7,8-tetrahydro-2H-indeno[4,5-b]furan (Aromatic Cyclized Compound) which can be used as an intermediate to make 2,2,6,6,7,8,8-heptamethyldecahydro-2H-indeno[4,5-b]furan (Musk Indenofuran). The present disclosure also relates to an anaerobic process of making various intermediate compounds which can be used to make Aromatic Cyclized Compound or Musk Indenofuran. The present disclosure also relates to phosphine derivatives and the process of making them from the Aromatic Cyclized Compound.

### Description of Related Art

IFF Amber Xtreme™ fragrance is a popular and powerful amber woody fragrance ingredient that can provide extraordinary performance to many fragrance types. It can be prepared using dihydro indomuscone (1,1,2,3,3-pentamethyloctahydro-4H-inden-4-one) as the starting material. However, the process takes five steps. Accordingly, there remains a need to optimize the manufacturing process.

### BRIEF SUMMARY OF THE DISCLOSURE

The present disclosure provides a process for making Aromatic Cyclized Compound. The process comprises: cyclizing a starting material in the presence of oxygen, an acid catalyst and a solvent in a reaction zone to form 2,2,6,6,7,8,8-heptamethyl-3,6,7,8-tetrahydro-2H-indeno[4,5-b]furan (Aromatic Cyclized Compound), wherein the acid catalyst is a homogeneous acid catalyst with pKa of no more than zero and/or a heterogeneous acid catalyst, and the starting material is 1,1,2,3,3-pentamethyl-5-(2-methylallyl)-1,2,3,5,6,7-hexahydro-4H-inden-4-one (Methallyl Indomuscone) and/or 5-(2-hydroxy-2-methylpropyl)-1,1,2,3,3-pentamethyl-1,2,3,5,6,7-hexahydro-4H-inden-4-one (Hydroxyisobutyl Indomuscone).

The present disclosure also provides a process for making intermediate compound(s) which can be used to make Aromatic Cyclized Compound or Musk Indenofuran. The process comprises: transforming a starting material in the presence of an acid catalyst and a solvent in a reaction zone into an intermediate compound selected from the group consisting of 1,1,2,3,3-pentamethyl-5-(2-methylallyl)-2,3-dihydro-1H-inden-4-ol (Methallyl Phenol Compound), 2,2,6,6,7,8,8-heptamethyl-3,4,5,6,7,8-hexahydro-2H-indeno[4,5-b]furan (Endocyclic Diene I), 2,2,6,6,7,8,8-heptamethyl-3,3a,4,6,7,8-hexahydro-2H-indeno[4,5-b]furan (Endocyclic Diene II), and mixtures thereof, wherein the transforming process is conducted under substantial absence of oxygen, the acid catalyst is a homogeneous acid catalyst with pKa of no more than zero and/or a heterogeneous acid catalyst, and the starting material is 1,1,2,3,3-pentamethyl-5-(2-methylallyl)-1,2,3,5,6,7-hexahydro-4H-inden-4-one (Methallyl Indomuscone) and/or 5-(2-hydroxy-2-methylpropyl)-1,1,2,3,3-pentamethyl-1,2,3,5,6,7-hexahydro-4H-inden-4-one (Hydroxyisobutyl Indomuscone).

The present disclosure also provides a process for making Musk Indenofuran. The process comprises: (a) alkylating Indomuscone with methallyl chloride under conditions effective to produce Methallyl Indomuscone, (b) cyclizing Methallyl Indomuscone under conditions effective to produce Aromatic Cyclized Compound, and (c) hydrogenating Aromatic Cyclized Compound under conditions effective to produce Musk Indenofuran.

The present disclosure also provides a process for making Musk Indenofuran. The process comprises: (a) alkylating Indomuscone with isobutylene oxide under conditions effective to produce Hydroxyisobutyl Indomuscone, (b) cyclizing Hydroxyisobutyl Indomuscone under conditions effective to produce Aromatic Cyclized Compound, and (c) hydrogenating Aromatic Cyclized Compound under conditions effective to produce Musk Indenofuran.

The present disclosure also provides a process for making Musk Indenofuran. The process comprises: contacting Hydroxyisobutyl Indomuscone with a hydrogenation catalyst in the presence of a solvent and hydrogen (H₂) in a reaction zone to produce a product mixture comprising Musk Indenofuran.

The present disclosure also provides a phosphine derivative compound of structural formula (X)

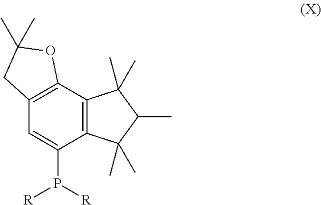

wherein R is selected from the group consisting of alkyl group, cycloalkyl group, phenyl group, substituted phenyl group, and perfluorinated alkyl group.

The present disclosure further provides a process for making the phosphine derivative compound of structural formula (X). The process comprises: (a) brominating Aromatic Cyclized Compound in the presence of a brominating agent and a solvent to form 5-bromo-2,2,6,6,7,8,8-heptamethyl-3,6,7,8-tetrahydro-2H-indeno[4,5-b]furan (Aromatic Bromide Compound), (b) contacting Aromatic Bromide Compound with a lithiating agent in the presence of a solvent in a reaction zone to form a reaction mixture, and (c) adding a phosphine chloride compound of formula R₂PCI to the reaction mixture to form the phosphine derivative compound of structural formula (X), wherein R is selected from the group consisting of alkyl group, cycloalkyl group, phenyl group, substituted phenyl group, and perfluorinated alkyl group.

### DETAILED DESCRIPTION

The foregoing general description and the following detailed description are exemplary and explanatory only and are not restrictive of the invention, as defined in the appended claims. Other features and benefits of any one or more of the embodiments will be apparent from the following detailed description, and from the claims.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8”, “3 to 10”, “2 to 7”, “1.5 to 6”, “3.4 to 7.8”, “1 to 2 and 7-10”, “2 to 4 and 6 to 9”, “1 to 3.6 and 7.2 to 8.9”, “1-5 and 10”, “2 and 8 to 10”, “1.5-4 and 8”, and the like.

The present disclosure illustratively described herein suitably may be practiced in the absence of any element or elements, limitation or limitations that are not specifically disclosed herein. While compositions and methods are described herein in terms of “comprising” various components or steps, the compositions and methods also can “consist essentially of” or “consist of” the various components or steps, unless stated otherwise.

A person of ordinary skill in the art appreciates that some chemical compounds in this disclosure have chiral center, carbon-carbon double bond, and/or cyclic structure. Unless explicitly indicated, a chemical compound in this disclosure includes its stereoisomers, such as enantiomers and diastereomers.

Before addressing details of embodiments described below, some terms are defined or clarified.

The term “Indomuscone”, as used herein, is the chemical compound named 1,1,2,3,3-pentamethyl-1,2,3,5,6,7-hexahydro-4H-inden-4-one and represented by the following structural formula (I):

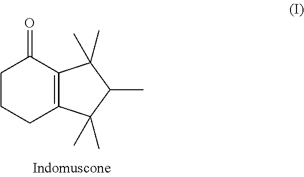

The term “Methallyl Indomuscone”, as used herein, is the chemical compound named 1,1,2,3,3-pentamethyl-5-(2-methylallyl)-1,2,3,5,6,7-hexahydro-4H-inden-4-one and represented by the following structural formula (II):

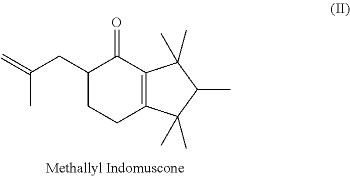 [SMILES: C=C(C)CC1CCC2=C(C1=O)C(C)(C)C(C)C2(C)C]

The term “Aromatic Cyclized Compound”, as used herein, is the chemical compound named 2,2,6,6,7,8,8-heptamethyl-3,6,7,8-tetrahydro-2H-indeno[4,5-b]furan and represented by the following structural formula (III):

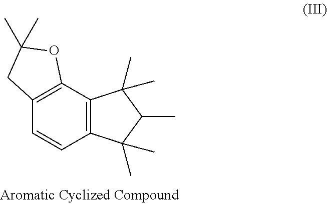

The term “Musk Indenofuran”, as used herein, is the chemical compound named 2,2,6,6,7,8,8-heptamethyldecahydro-2H-indeno[4,5-b]furan and represented by the following structural formula (IV):

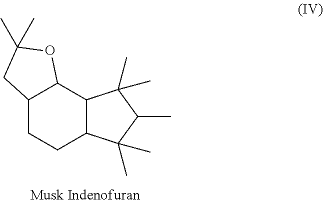

The term “Hydroxyisobutyl Indomuscone”, as used herein, is the chemical compound named 5-(2-hydroxy-2-methylpropyl)-1,1,2,3,3-pentamethyl-1,2,3,5,6,7-hexahydro-4H-inden-4-one and represented by the following structural formula (V):

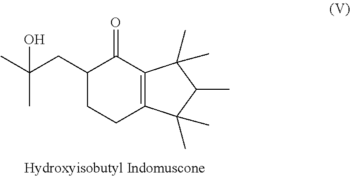 [SMILES: CC1C(C)(C)C2=C(C(=O)C(CC(C)(C)O)CC2)C1(C)C]

The term “Methallyl Phenol Compound”, as used herein, is the chemical compound named 1,1,2,3,3-pentamethyl-5-(2-methylallyl)-2,3-dihydro-1H-inden-4-ol and represented by the following structural formula (VI):

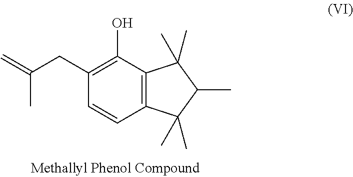 [SMILES: C=C(C)Cc1ccc2c(c1O)C(C)(C)C(C)C2(C)C]

The term “Endocyclic Diene I”, as used herein, is the chemical compound named 2,2,6,6,7,8,8-heptamethyl-3,4,5,6,7,8-hexahydro-2H-indeno[4,5-b]furan and represented by the following structural formula (VII):

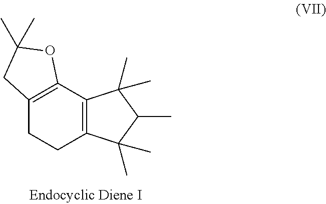

The term “Endocyclic Diene II”, as used herein, is the chemical compound named 2,2,6,6,7,8,8-heptamethyl-3,3a,4,6,7,8-hexahydro-2H-indeno[4,5-b]furan and represented by the following structural formula (VIII):

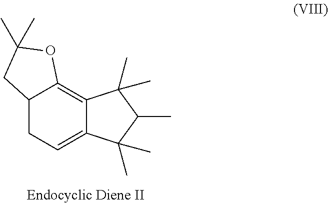

The term “Aromatic Bromide Compound”, as used herein, is the chemical compound named 5-bromo-2,2,6,6,7,8,8-heptamethyl-3,6,7,8-tetrahydro-2H-indeno[4,5-b]furan and represented by the following structural formula (IX):

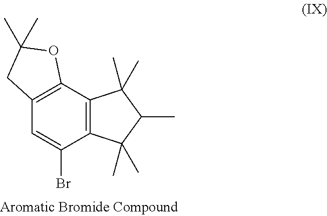

The term “heterogeneous acid catalyst” or “solid acid catalyst”, as used herein, means a solid catalyst comprising Brønsted acid (or protic) sites. Brønsted acid site is a site with an ionizable hydrogen atom. The terms “heterogeneous acid catalyst” and “solid acid catalyst” can be used interchangeably in this disclosure.

The present disclosure provides a process for making 2,2,6,6,7,8,8-heptamethyl-3,6,7,8-tetrahydro-2H-indeno[4,5-b]furan (Aromatic Cyclized Compound). The process comprises cyclizing a starting material in the presence of oxygen, an acid catalyst and a solvent in a reaction zone to form the Aromatic Cyclized Compound, wherein the acid catalyst is a homogeneous acid catalyst with pKa of no more than zero and/or a heterogeneous acid catalyst, and the starting material is 1,1,2,3,3-pentamethyl-5-(2-methylallyl)-1,2,3,5,6,7-hexahydro-4H-inden-4-one (Methallyl Indomuscone) and/or 5-(2-hydroxy-2-methylpropyl)-1,1,2,3,3-pentamethyl-1,2,3,5,6,7-hexahydro-4H-inden-4-one (Hydroxyisobutyl Indomuscone).

Alternatively, the present disclosure provides a process for making an intermediate compound to the Aromatic Cyclized Compound. The process comprises transforming a starting material in the presence of an acid catalyst and a solvent in a reaction zone into an intermediate compound selected from the group consisting of 1,1,2,3,3-pentamethyl-5-(2-methylallyl)-2,3-dihydro-1H-inden-4-ol (Methallyl Phenol Compound), 2,2,6,6,7,8,8-heptamethyl-3,4,5,6,7,8-hexahydro-2H-indeno[4,5-b]furan (Endocyclic Diene I), 2,2,6,6,7,8,8-heptamethyl-3,3a,4,6,7,8-hexahydro-2H-indeno[4,5-b]furan (Endocyclic Diene II), and mixtures thereof, wherein the transforming process is conducted under substantial absence of oxygen, the acid catalyst is a homogeneous acid catalyst with pKa of no more than zero and/or a heterogeneous acid catalyst, and the starting material is 1,1,2,3,3-pentamethyl-5-(2-methylallyl)-1,2,3,5,6,7-hexahydro-4H-inden-4-one (Methallyl Indomuscone) and/or 5-(2-hydroxy-2-methylpropyl)-1,1,2,3,3-pentamethyl-1,2,3,5,6,7-hexahydro-4H-inden-4-one (Hydroxyisobutyl Indomuscone).

In some embodiments, the starting material is the Methallyl Indomuscone. In some embodiments, the starting material is the Hydroxyisobutyl Indomuscone. In some embodiments, there is no noble metal catalyst, such as palladium (Pd) catalyst, present in the reaction zone.

pKa is a concept well known in the art. It is the negative base 10 logarithm of the acid dissociation constant Ka, that is, pka=−log₁₀Ka. pKa in this disclosure means the pKa value of the corresponding acid in water solution at 25° C. In some embodiments, the acid catalyst is a homogeneous acid catalyst with pKa of no more than zero, or no more than-1, or no more than-2. In some embodiments, the homogeneous acid catalyst is selected from the group consisting of p-toluenesulfonic acid (pTsOH), methanesulfonic acid (MSA), sulfuric acid, triflic acid (trifluoromethanesulfonic acid, TfOH), trifluoromethanesulfonimide ((CF₃SO₂)₂NH), trifluoromethanesulfonamide (CF₃SO₂NH₂), and mixtures thereof. In some embodiments, the homogeneous acid catalyst is selected from the group consisting of p-toluenesulfonic acid (PTSA), methanesulfonic acid (MSA), and mixtures thereof.

In some embodiments, the heterogeneous acid catalyst is selected from the group consisting of alumina, silica-alumina, zeolite, silico-alumino-phosphate, aluminophosphate, sulfated zirconia, zirconia (zirconium dioxide), sulfonic acid resin, and mixtures thereof. In some embodiments, the silica-alumina catalyst is amorphous. In some embodiments, the heterogeneous acid catalyst has a porous structure. In some embodiments, the heterogeneous acid catalyst is selected from the group consisting of zeolite, sulfonic acid resin, and mixtures thereof. The term “sulfonic acid resin”, as used herein, means a resin (such as a polystyrene resin) functionalized with a p-toluenesulfonic acid end group. Examples of sulfonic acid resin include Amberlyst acidic cation exchange resins such as Amberlyst 15 resin, Amberlyst 16 resin and Amberlyst 20 resin. In some embodiments, the heterogeneous acid catalyst is zeolite. In this disclosure, the zeolite comprises Brønsted acid (or protic) sites. Examples of suitable zeolite include H-Beta zeolite and H-USY zeolite.

In some embodiments, the acid catalyst is the homogeneous acid catalyst, and the amount of the homogeneous acid catalyst is at least 0.01 mol %, or at least 0.1 mol %, or at least 0.5 mol %, or at least 1 mol %, or at least 5 mol %, or at least 10 mol %, or at least 15 mol % based on the mole amount of the starting material. In some embodiments, the amount of the homogeneous acid catalyst is no more than 10 times, or 5 times, or 2 times, or 1 times the mole amount of the starting material.

In some embodiments, the acid catalyst is the heterogeneous acid catalyst, and the amount of the heterogeneous acid catalyst is at least 1 wt %, or at least 5 wt %, or at least 10 wt %, or at least 20 wt %, or at least 30 wt %, or at least 40 wt %, or at least 50 wt % based on the weight of the starting material. In some embodiments, the amount of the heterogeneous acid catalyst is no more than 50 times, or 10 times, or 5 times, or 2 times the weight of the starting material.

Examples of the solvent include toluene, xylene, alcohols, ethers, and their combinations. Examples of alcohol include methanol, ethanol, 1-propanol, isopropanol, butanol and its isomers, pentanol and its isomers, and their combinations. Examples of ether include THF, dioxane, anisole, and their combinations. In some embodiments, the solvent is substantially free of acetonitrile. In some embodiments, the solvent is substantially free of water. In some embodiments, the solvent comprises no more than 20 wt %, or no more than 10 wt %, or no more than 5 wt %, or no more than 2 wt %, or no more than 1 wt % of acetonitrile, based on the total weight of the solvent. In some embodiments, the solvent comprises no more than 10 wt %, or no more than 5 wt %, or no more than 2 wt %, or no more than 1 wt % of water, based on the total weight of the solvent.

In some embodiments, the concentration of the starting material in the solvent is from about 0.05 M (mol/L) to about 5 M, or from about 0.1 M to about 3 M, or from about 0.2 M to about 2 M. In some embodiments, the concentration of the starting material in the solvent is at least 0.01 M, or at least 0.02 M, or at least 0.05 M, or at least 0.1 M, or at least 0.2 M, or at least 0.3 M, or at least 0.4 M, or at least 0.5 M. In some embodiments, the concentration of the starting material in the solvent is no more than 10 M, or no more than 5 M, or no more than 4 M, or no more than 3 M, or no more than 2 M, or no more than 1 M.

In some embodiments, the acid catalyst is the homogeneous acid catalyst, and the cyclization or transformation (into an intermediate compound) reaction is conducted at a temperature (reaction temperature, or temperature in the reaction zone) of from about 20° C. to about 120° C., or from about 30° C. to about 110° C., or from about 40° C. to about 100° C., or from about 50° C. to about 90° C. In some embodiments, the reaction temperature is at least 10° C., or at least 20° C., or at least 30° C., or at least 40° C., or at least 50° C., or at least 60° C. In some embodiments, the reaction temperature is no more than 150° C., or no more than 140° C., or no more than 130° C., or no more than 120° C., or no more than 110° C., or no more than 100° C., or no more than 90° C., or no more than 80° C.

In some embodiments, the acid catalyst is the heterogeneous acid catalyst, and the cyclization or transformation (into an intermediate compound) reaction is conducted at a temperature (reaction temperature, or temperature in the reaction zone) of from about 70° C. to about 150° C., or from about 80° C. to about 140° C., or from about 90° C. to about 130° C., or from about 100° C. to about 120° C. In some embodiments, the reaction temperature is at least 50° C., or at least 60° C., or at least 70° C., or at least 80° C., or at least 90° C., or at least 100° C. In some embodiments, the reaction temperature is no more than 200° C., or no more than 180° C., or no more than 170° C., or no more than 160° C., or no more than 150° C., or no more than 140° C., or no more than 130° C., or no more than 120° C.

The reaction zone pressure is not critical for the cyclization or transformation reaction. The cyclization or transformation reaction can be conducted under atmospheric pressure or under pressures less than or greater than atmospheric pressure. In some embodiments, the cyclization reaction is conducted under ambient atmosphere (i.e., air).

The cyclizing or transforming process time (cyclization or transformation reaction time) can range from about 1 hr (hour) to about 72 hrs (hours), or from about 6 hrs to about 48 hrs, or from about 8 hrs to about 36 hrs. In some embodiments, the cyclization or transformation reaction time is at least 1 hr, or at least 2 hrs, or at least 4 hrs, or at least 6 hrs, or at least 8 hrs, or at least 10 hrs, or at least 12 hrs, or at least 14 hrs, or at least 16 hrs, or at least 18 hrs, or at least 20 hrs, or at least 22 hrs, or at least 24 hrs. In some embodiments, the cyclization or transformation reaction time is no more than 7 days, or no more than 6 days, or no more than 5 days, or no more than 4 days, or no more than 72 hrs, or no more than 60 hrs, or no more than 54 hrs, or no more than 48 hrs, or no more than 42 hrs, or no more than 36 hrs, or no more than 30 hrs, or no more than 24 hrs.

The cyclization reaction is conducted in the presence of oxygen. Oxygen may be in the form of pure oxygen, oxygen mixed with an inert gas such as nitrogen, or oxygen present in air. In some embodiments, the cyclization reaction can be first conducted under nitrogen (i.e., in the absence of oxygen) and then exposed to oxygen.

The desired product Aromatic Cyclized Compound can be separated and recovered by methods known in the art such as distillation and chromatography. In some embodiments, the yield of Aromatic Cyclized Compound is at least 50%, or at least 55%, or at least 60%, or at least 65%, or at least 70%.

The transformation reaction is conducted under substantial absence of oxygen. By “substantial absence of oxygen” means the amount of oxygen present or fed in the reaction zone is no more than 40 mol %, or no more than 30 mol %, or no more than 20 mol %, or no more than 10 mol %, or no more than 5 mol % based on the mole amount of the starting material. In some embodiments, the transformation reaction can be conducted under an inert gas such as nitrogen.

In some embodiments, the intermediate compound(s) can be further transformed into the Aromatic Cyclized Compound. The Methallyl Phenol Compound can be transformed into the Aromatic Cyclized Compound in the presence of an acid catalyst and a solvent in a reaction zone. In some embodiments, the acid catalyst and/or the solvent are the same as the ones used in the transformation (into intermediate compound) reaction. The endocyclic diene (Endocyclic Diene I and/or Endocyclic Diene II) can be transformed into the Aromatic Cyclized Compound in the presence of oxygen and a solvent in a reaction zone. In some embodiments, the solvent is the same as the one used in the transformation (into intermediate compound) reaction.

In some embodiments, the intermediate compound(s) is further transformed in the presence of oxygen into Aromatic Cyclized Compound. In some embodiments, after transforming a starting material in the presence of an acid catalyst and a solvent in a reaction zone into an intermediate compound, oxygen (e.g., an oxygen-containing gas such as air) is introduced into the reaction zone, and the intermediate compound is further transformed in situ in the presence of oxygen, the acid catalyst and the solvent into Aromatic Cyclized Compound without separating or purifying the intermediate compound from the reaction mixture and without removal of the acid catalyst and the solvent from the reaction zone.

The reaction temperature of the further transformation (from intermediate compound into Aromatic Cyclized Compound) can be in the same range as the cyclization or transformation (into an intermediate compound) reaction temperature described in this disclosure.

In some embodiments, the intermediate compound generated in the transforming process comprises an endocyclic diene compound (Endocyclic Diene I and/or Endocyclic Diene II), and the endocyclic diene compound can be hydrogenated in the presence of a hydrogenation catalyst, a solvent and hydrogen under effective conditions to form 2,2,6,6,7,8,8-heptamethyldecahydro-2H-indeno[4,5-b]furan (Musk Indenofuran). In some embodiments, the transforming process is performed in a reaction zone in the presence of a zeolite catalyst and a solvent selected from the group consisting of alcohols, ethers, and mixtures thereof. After the transforming process, a hydrogenation catalyst is fed into the reaction zone, and the hydrogenating process can be performed in situ without separating or purifying the endocyclic diene compound from the reaction mixture and without removal of the zeolite catalyst and the solvent from the reaction zone. Alternatively, the transforming process is performed with a heterogeneous acid catalyst, and after the transforming process, the heterogeneous acid catalyst is removed (e.g., by filtration) from the reaction mixture, a hydrogenation catalyst is fed into the reaction zone, and the hydrogenating process can be performed in situ without separating the endocyclic diene compound and the solvent from the reaction mixture.

Methallyl Indomuscone can be synthesized through alkylation of Indomuscone with methallyl chloride (CH₂═C(CH₃)CH₂Cl). In some embodiments, the process comprises alkylating Indomuscone with methallyl chloride in the presence of a strong base and a solvent in a reaction zone to produce a product mixture comprising Methallyl Indomuscone. Examples of the strong base include sodium amide (NaNH₂), lithium diisopropylamide (LiN(CH(CH₃)₂)₂), and their combinations. Examples of the solvent include toluene, THF, and their combinations. In some embodiments, the mole ratio of Indomuscone to methallyl chloride fed into the reaction zone is from about 1:1 to about 1:5, or from about 1:1 to about 1:1.5. In some embodiments, the mole ratio of Indomuscone to strong base fed into the reaction zone is from about 1:1 to about 1:5, or from about 1:1 to about 1:1.5. The reaction temperature can be in a range of from about 25° C. to about 150° C., or from about 40° C. to about 130° C., or from about 40° C. to about 80° C., or from about 90° C. to about 130° C. The reaction time can be in a range of from about 1 hr to about 36 hrs, or from about 4 hrs to about 30 hrs, or from about 6 hrs to about 18 hrs, or from about 12 hrs to about 30 hrs. In some embodiments, the yield of Methallyl Indomuscone is at least 50%, or at least 55%, or at least 60%.

Hydroxyisobutyl Indomuscone can be synthesized through alkylation of Indomuscone with isobutylene oxide. In some embodiments, the process comprises alkylating Indomuscone with isobutylene oxide in the presence of a strong base and a solvent in a reaction zone to produce a product mixture comprising Hydroxyisobutyl Indomuscone. Examples of the strong base include sodium amide (NaNH₂), lithium diisopropylamide (LiN(CH(CH₃)₂)₂), and their combinations. Examples of the solvent include toluene, THF, and their combinations. In some embodiments, the mole ratio of Indomuscone to isobutylene oxide fed into the reaction zone is from about 1:1 to about 1:5, or from about 1:1 to about 1:1.5. In some embodiments, the mole ratio of Indomuscone to strong base fed into the reaction zone is from about 1:1 to about 1:5, or from about 1:1 to about 1:1.5. The reaction temperature can be in a range of from about 25° C. to about 150° C., or from about 25° C. to about 120° C., or from about 25° C. to about 100° C., or from about 25° C. to about 80° C., or from about 40° C. to about 80° C., or from about 50° C. to about 70° C., or from about 35° C. to about 70° C. The reaction time can be in a range of from about 1 hr to about 24 hrs, or from about 2 hrs to about 20 hrs, or from about 3 hrs to about 16 hrs, or from about 6 hrs to about 20 hrs, or from about 8 hrs to about 16 hrs. In some embodiments, the yield of Hydroxyisobutyl Indomuscone is at least 55%, or at least 60%, or at least 65%, or at least 70%, or at least 75%.

In some embodiments, Aromatic Cyclized Compound is hydrogenated to form Musk Indenofuran. In some embodiments, the process comprises hydrogenating Aromatic Cyclized Compound in the presence of a hydrogenation catalyst, a solvent and hydrogen (H₂) in a reaction zone to produce a product mixture comprising Musk Indenofuran. In some embodiments, the hydrogenation catalyst is a heterogeneous catalyst comprising rhodium metal loaded on a support. In some embodiments, the support is carbon such as an activated carbon. In some embodiments, the content of the rhodium element is from 1 wt % to 20 wt %, or from 1 wt % to 15 wt %, or from 2 wt % to 10 wt %, based on the total weight of the catalyst. Examples of the solvent include alcohols, ethers, and their combinations. Examples of alcohol include methanol, ethanol, 1-propanol, isopropanol, butanol and its isomers, pentanol and its isomers, and their combinations. Examples of ether include THF, dioxane, and their combinations. In some embodiments, the solvent is ethanol and/or THF. In some embodiments, the solvent is ethanol. The hydrogenation reaction temperature can be in a range of from about 25° C. to about 160° C., or from about 100° C. to about 160° C., or from about 130° C. to about 160° C., or from about 130° C. to about 150° C. In some embodiments, the hydrogenating process can be conducted under 10-100 bars of H₂, or 20-60 bars of H₂. The reaction time can be in a range of from about 1 hr to about 48 hrs, or from about 6 hrs to about 36 hrs, or from about 10 hrs to about 24 hrs. In some embodiments, the yield of Musk Indenofuran is at least 50%, or at least 60%, or at least 70%, or at least 80%.

In some embodiments, the cyclizing process and the hydrogenating process can be conducted in the same reaction zone. For example, the cyclizing process can be conducted in a reaction zone in the presence of oxygen, a zeolite catalyst and a solvent selected from the group consisting of alcohols (e.g., ethanol), ethers (e.g., THF), and mixtures thereof to generate a product mixture comprising Aromatic Cyclized Compound. After the cyclizing process, a hydrogenation catalyst can be fed into the reaction zone, and the hydrogenating process can be conducted in situ without separating or purifying the Aromatic Cyclized Compound from the product mixture and without removal of the zeolite catalyst and the solvent from the reaction zone. In some embodiments, the zeolite catalyst is removed from the reaction zone after the cyclizing process and before the addition of the hydrogenation catalyst into the reaction zone.

The present disclosure also provides a process for making Musk Indenofuran. The process comprises: (a) alkylating Indomuscone with methallyl chloride under conditions effective to produce Methallyl Indomuscone, (b) cyclizing Methallyl Indomuscone under conditions effective to produce Aromatic Cyclized Compound, and (c) hydrogenating Aromatic Cyclized Compound under conditions effective to produce Musk Indenofuran. In some embodiments, the respective conditions effective to produce Methallyl Indomuscone, Aromatic Cyclized Compound, or Musk Indenofuran have been described in this disclosure.

The present disclosure also provides a process for making Musk Indenofuran. The process comprises: (a) alkylating Indomuscone with isobutylene oxide under conditions effective to produce Hydroxyisobutyl Indomuscone, (b) cyclizing Hydroxyisobutyl Indomuscone under conditions effective to produce Aromatic Cyclized Compound, and (c) hydrogenating Aromatic Cyclized Compound under conditions effective to produce Musk Indenofuran. In some embodiments, the respective conditions effective to produce Hydroxyisobutyl Indomuscone, Aromatic Cyclized Compound, or Musk Indenofuran have been described in this disclosure.

The present disclosure also provides a one-pot process for making Musk Indenofuran by using Hydroxyisobutyl Indomuscone as the starting material. The process comprises contacting Hydroxyisobutyl Indomuscone with a hydrogenation catalyst in the presence of a solvent and hydrogen (H₂) in a reaction zone to produce a product mixture comprising Musk Indenofuran. In this embodiment, Hydroxyisobutyl Indomuscone is cyclized and hydrogenated in the presence of a hydrogenation catalyst to form Musk Indenofuran. The process can be conducted in a single reaction zone without separating or purifying an intermediate compound generated during the process.

In some embodiments, the hydrogenation catalyst is a heterogeneous catalyst. In some embodiments, the heterogeneous catalyst is selected from the group consisting of ruthenium (Ru) catalyst, rhodium (Rh) catalyst, palladium (Pd) catalyst, platinum (Pt) catalyst, and mixtures thereof. The ruthenium catalyst is a ruthenium-containing catalyst wherein ruthenium can be present as a mixture of ruthenium in metal form and ruthenium compound(s), that is, ruthenium can be in an oxidation state of 0, I, II or III. A typical form of ruthenium here is as a metal nanoparticle or as an oxide. The rhodium catalyst is a rhodium-containing catalyst wherein rhodium can be present as a mixture of rhodium in metal form and rhodium compound(s), that is, rhodium can be in an oxidation state of 0 or I. A typical form of rhodium here is as a metal nanoparticle or as an oxide. The palladium catalyst is a palladium-containing catalyst wherein palladium can be present as a mixture of palladium in metal form and palladium compound(s), that is, palladium can be in an oxidation state of 0 or II. A typical form of palladium here is as a metal nanoparticle or as an oxide. The platinum catalyst is a platinum-containing catalyst wherein platinum is present as a mixture of platinum in metal form and platinum compound(s), that is, platinum can be in an oxidation state of 0 or II. A typical form of platinum here is as a metal nanoparticle or as an oxide.

In some embodiments, the heterogeneous hydrogenation catalyst is loaded on a catalyst support. In some embodiments, the support is carbon such as an activated carbon. Other suitable supports include alumina, silica, and mixtures thereof. In some embodiments, the content of the ruthenium element is from 1 wt % to 20 wt %, or from 1 wt % to 15 wt %, or from 2 wt % to 10 wt %, based on the total weight of the ruthenium catalyst and support. In some embodiments, the content of the rhodium element, the palladium element, or the platinum element is from 1 wt % to 20 wt %, or from 1 wt % to 15 wt %, or from 2 wt % to 10 wt %, based on the total weight of the corresponding catalyst and support.

In some embodiments, the amount of the ruthenium catalyst is such that the total amount of ruthenium element contained in the catalyst is at least 0.05 mol %, or at least 0.1 mol %, or at least 0.2 mol %, or at least 0.3 mol %, or at least 0.4 mol % based on the total molar amount of the Hydroxyisobutyl Indomuscone starting material. In some embodiments, the amount of the ruthenium catalyst is such that the total amount of ruthenium element contained in the catalyst is no more than 5 mol %, or no more than 4 mol %, or no more than 3 mol %, or no more than 2 mol %, or no more than 1 mol %, or no more than 0.8 mol %, or no more than 0.6 mol % based on the total molar amount of the Hydroxyisobutyl Indomuscone starting material.

In some embodiments, the amount of the rhodium catalyst, the palladium catalyst, or the platinum catalyst is such that the total amount of rhodium element, palladium element, or platinum element contained in the corresponding catalyst is at least 0.05 mol %, or at least 0.1 mol %, or at least 0.2 mol %, or at least 0.3 mol %, or at least 0.4 mol % based on the total molar amount of the Hydroxyisobutyl Indomuscone starting material. In some embodiments, the amount of the rhodium catalyst, the palladium catalyst, or the platinum catalyst is such that the total amount of rhodium element, palladium element, or platinum element contained in the corresponding catalyst is no more than 5 mol %, or no more than 4 mol %, or no more than 3 mol %, or no more than 2 mol %, or no more than 1 mol %, or no more than 0.8 mol %, or no more than 0.6 mol % based on the total molar amount of the Hydroxyisobutyl Indomuscone starting material.

Examples of the solvent include alcohols, ethers, and their combinations. Examples of alcohol include methanol, ethanol, 1-propanol, isopropanol, butanol and its isomers, pentanol and its isomers, and their combinations. Examples of ether include THF, dioxane, and their combinations. In some embodiments, the solvent is ethanol and/or THF. In some embodiments, the solvent is ethanol.

The reaction temperature of the one-pot process of making Musk Indenofuran can be in a range of from about 70° C. to about 250° C., or from about 80° C. to about 220° C., or from about 90° C. to about 200° C., or from about 100° C. to about 180° C., or from about 120° C. to about 160° C. In some embodiments, the one-pot process can be conducted under 10-100 bars of H₂, or 20-60 bars of H₂. The reaction time can be in a range of from about 1 hr to about 48 hrs, or from about 6 hrs to about 36 hrs, or from about 10 hrs to about 24 hrs. In some embodiments, the yield of Musk Indenofuran is at least 55%, or at least 60%, or at least 65%.

During the one-pot process, intermediate compounds such as Endocyclic Diene I and/or Endocyclic Diene II can be formed. The process is conducted by using Hydroxyisobutyl Indomuscone as the starting material to make Musk Indenofuran without separating or purifying any intermediate compounds. At the end of the reaction, the desired product Musk Indenofuran can be separated and recovered by methods known in the art such as distillation and chromatography.

The present disclosure also provides a phosphine derivative compound of structural formula (X)

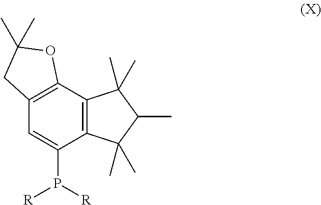

wherein R is selected from the group consisting of alkyl group, cycloalkyl group, phenyl group, substituted phenyl group, and perfluorinated alkyl group. In some embodiments, R is a phenyl group (Ph). In some embodiments, R is a cycloalkyl group selected from the group consisting of cyclohexyl group, cyclopentyl group, cyclobutyl group, and cyclopropyl group. In some embodiments, R is a cyclohexyl group. In some embodiments, R is a cyclopentyl group. In some embodiments, R is a linear alkyl group. In some embodiments, R is an alkyl group selected from the group consisting of methyl group, ethyl group, isopropyl group, and tert-butyl group (′Bu). In some embodiments, R is an isopropyl group. In some embodiments, R is a tert-butyl group.

The phosphine derivative compound of structural formula (X) (“Phosphine Derivative Compound”) can be used as a ligand to form a metal-phosphine complex which can be used as a catalyst for, for instance, carbon-carbon, carbon-amine or carbon-oxygen cross coupling reactions.

The phosphine derivative compound of structural formula (X) can be synthesized using the Aromatic Cyclized Compound as a starting material, as shown in Scheme 1.

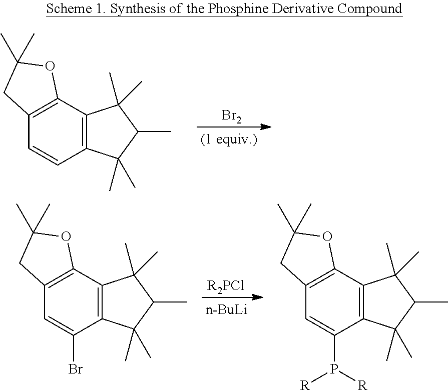

The present disclosure also provides a process for making the phosphine derivative compound of structural formula (X). The process comprises: (a) brominating Aromatic Cyclized Compound in the presence of a brominating agent and a solvent to form 5-bromo-2,2,6,6,7,8,8-heptamethyl-3,6,7,8-tetrahydro-2H-indeno[4,5-b]furan (Aromatic Bromide Compound), (b) contacting Aromatic Bromide Compound with a lithiating agent in the presence of a solvent in a reaction zone to form a reaction mixture, and (c) adding a phosphine chloride compound of formula R₂PCI to the reaction mixture to form the phosphine derivative compound of structural formula (X) (Phosphine Derivative Compound).

The process comprises a bromination reaction (step (a)) and a phosphination reaction (steps (b) and (c)). In the bromination reaction, in some embodiments, the brominating agent is selected from the group consisting of bromine (Br₂), N-bromosuccinimide (NBS), 1,3-dibromo-5,5-dimethylhydantoin (DBDMH), N-bromobenzamide, pyridinium hydrobromide perbromide (PyHBr₃), and mixtures thereof. In some embodiments, the brominating agent is selected from the group consisting of bromine (Br₂), N-bromosuccinimide (NBS), 1,3-dibromo-5,5-dimethylhydantoin (DBDMH), and mixtures thereof. In some embodiments, the amount of the brominating agent for the reaction is from about 5 to about 1 stoichiometric equivalent, or from about 2 to about 1 stoichiometric equivalent, or from about 1.5 to about 1 stoichiometric equivalent, or from about 1.2 to about 1 stoichiometric equivalent, or from about 1.1 to about 1 stoichiometric equivalent. Examples of the solvent for the bromination reaction include dichloromethane, hexane, acetonitrile, ethers such as diethyl ether, tert-butyl methyl ether and 1,4-dioxane, n-pentane, cyclohexane, and their combinations. The bromination reaction temperature may be in the range of from about 0° C. to about 50° C. In some embodiments, the bromination reaction is carried out at room temperature. The bromination reaction time can be from 1 to 120 min (minutes), or from 5 to 60 min, or from 10 to 30 min. In some embodiments, the yield of Aromatic Bromide Compound is at least about 70%, or at least about 75%, or at least about 80%, or at least about 85%.

In the phosphination reaction, in some embodiments, the lithiating agent is selected from the group consisting of n-butyllithium (n-BuLi), phenyllithium (PhLi), tert-butyllithium (tert-BuLi), sec-butyllithium (sec-BuLi), a 1:1 mixture of n-BuLi and tert-BuONa (sodium tert-butoxide), lithium metal, and mixtures thereof. In some embodiments, the lithiating agent is n-BuLi. In some embodiments, the mole ratio of the lithiating agent to Aromatic Bromide Compound is from about 2:1 to about 1:1, or from about 1.5:1 to about 1:1, or about 1.25:1. Examples of the solvent for the phosphination reaction include THF, hexane, diethyl ether, tert-butyl methyl ether, 1,4-dioxane, n-pentane, cyclohexane, and their combinations. In some embodiments, the temperature for the contacting step (b) is from about −90° C. to about 25° C., or from about −90° C. to about 0° C., or from about −85° C. to about −10° C., or from about −80° C. to about −20° C., or from about 0° C. to about 25° C., or about −78° C. In some embodiments, the contact time (of the contacting step (b)) is about 5 to 60 min, or about 5 to 40 min, or about 10 to 30 min. In some embodiments, the reaction temperature of the step (c) is from about 10° C. to about 100° C., or from about 15° C. to about 80° C., or from about 20° C. to about 60° C., or from about 20° C. to about 40° C., or at about room temperature. In some embodiments, the mole ratio of the phosphine chloride compound fed into the reaction zone to Aromatic Bromide Compound fed into the reaction zone is from about 2:1 to about 1:1, or from about 1.5:1 to about 1:1, or from about 1.2:1 to about 1:1, or from about 1.1:1 to about 1:1. In some embodiments, the reaction time of step (c) is about 30 to 240 min, or about 60 to 180 min, or about 60 to 120 min. In some embodiments, the yield of the Phosphine Derivative Compound is at least about 20%, or at least about 25%, or at least about 30%.

Many aspects and embodiments have been described above and are merely exemplary and not limiting. After reading this specification, skilled artisans appreciate that other aspects and embodiments are possible without departing from the scope of the invention.

### EXAMPLES

The concepts described herein will be further described in the following examples, which do not limit the scope of the invention described in the claims.

### General

Glassware was dried in an oven at 175° C. before use. Cyclization reactions were performed in vials or round-bottom flasks equipped with magnetic stirrer and open to the air. Reagents and solvents were obtained from commercial sources and were used without further purification unless otherwise indicated. Products were characterised by GC-MS, ¹H- and ¹³C-NMR, and DEPT (distortionless enhancement by polarization transfer). Gas chromatographic analyses were performed in an instrument equipped with a 25 m capillary column of 5% phenylmethylsilicone. n-dodecane was used as an external standard. GC/MS analyses were performed on a spectrometer equipped with the same column as the GC and operated under the same conditions. ¹H, ¹³C and DEPT measurements were recorded in a 300 MHz instrument using CDCl₃ as a solvent, containing TMS as an internal standard.

### Example 1: Cyclization of Methallyl Indomuscone to Aromatic Cyclized Compound with Various Acid Catalysts

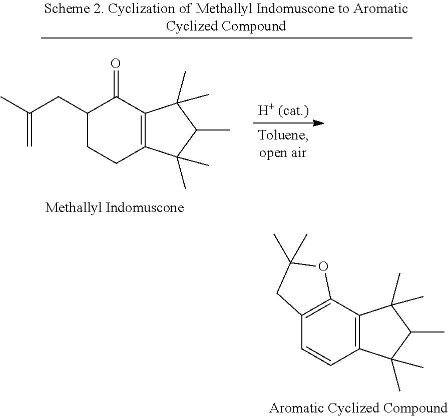

Cyclization of Methallyl Indomuscone with Homogeneous Acid Catalyst

In a 50 ml (milliliter) round-bottom flask equipped with a magnetic stir bar, the acid catalyst MSA (65 μl) was added to a solution of Methallyl Indomuscone (1.3 g, 5 mmol) in toluene (25 ml). The reaction mixture was open to the air and magnetically stirred at room temperature for 18 hours, and then concentrated under vacuum. Flash chromatography (100% hexane) generated 320 mg (25% yield) of the Aromatic Cyclized Compound as a yellow oil. Results are shown in Table 1.

Same process as above was conducted except that pTsOH (172 mg) was used as the acid catalyst. Flash chromatography (100% hexane) generated 256 mg (20% yield) of the Aromatic Cyclized Compound as a yellow oil. Results are shown in Table 1.

Cyclization of Methallyl Indomuscone with Zeolites

In a 25 ml round-bottom flask equipped with a magnetic stir bar and the corresponding zeolite (H-Beta CP-811 or H-USY CBV-720, 260 mg, previously calcined at 300° C. under vacuum), a solution of Methallyl Indomuscone (260 mg, 1 mmol) in toluene (5 ml) was added. The reaction mixture was open to the air and stirred at 110° C. under reflux. After 12 hours, an aliquot of the reaction mixture was dissolved in AcOEt (1 ml) and filtered through a 20 μm nylon filter and the resulting filtrate solution was analyzed by GC and GC-MS. Results are shown in Table 1.

TABLE 1

| | Acid Catalyst (amount relative | T | t | Conv | Yield |
|---|---|---|---|---|---|
| Entry | to Methallyl Indomuscone) | (° C.) | (hrs) | (%) | (%) |
| 1 | MSA (20 mol %) | 25 | 18 | 100 | 25 |
| 2 | pTsOH (20 mol %) | | | 90 | 20 |
| 3 | H-Beta (CP-811, 100 wt %) | 110 | 12 | 100 | 70 |
| 4 | H-USY (CBV-720, 100 wt %) | 110 | 12 | 100 | 50 |

Note:
In Tables, T means reaction temperature; t means reaction time; Conv means conversion.

### Example 2: Cyclization of Methallyl Indomuscone to Aromatic Cyclized Compound with pTsOH at Various Temperatures

In a 250 ml round-bottom flask equipped with a magnetic stir bar, the acid catalyst pTsOH (7 g) was added to a solution of Methallyl Indomuscone (52 g, 0.2 mol) in toluene (100 ml). The reaction mixture was open to the air and magnetically stirred at a certain temperature (25° C., 50° C. or 70° C.) for a certain reaction time. After cooling, the mixture was neutralized with 10% aqueous sodium hydrogen carbonate solution. The aqueous phase was extracted with hexane and washed with brine. The combined organic phases were dried over MgSO₄, filtered and concentrated under vacuum. Flash chromatography (100% hexane) was used for purifying and recovering the product Aromatic Cyclized Compound. Reaction conditions and results are shown in Table 2.

TABLE 2

| Entry | Solvent | T (° C.) | t (hrs) | Conv (%) | Yield (%) |
|---|---|---|---|---|---|
| 1 | Toluene | 70 | 24 | 100 | 70 |
| 2 | | 50 | 36 | | 45 |
| 3 | | 25 | 48 | | 20 |

### Example 3: Cyclization of Methallyl Indomuscone to Aromatic Cyclized Compound with pTsOH in Various Solvents

In a 8 ml vial equipped with a magnetic stir bar and containing the acid catalyst pTsOH (140 mg), a solution of Methallyl Indomuscone (1.04 g, 4 mmol) in a solvent (2 ml) was added. The reaction mixture was open to the air and stirred at a certain temperature for 24 hrs. After that time, the mixture was cooled and neutralized with 10% aqueous sodium hydrogen carbonate solution. The aqueous phase was extracted with hexane and washed with brine. The combined organic phases were dried over MgSO₄, filtered and concentrated under vacuum. An aliquot was dissolved in AcOEt (1 ml) and filtered through a 20 μm nylon filter, and the resulting filtrate solution was analyzed by GC and GC-MS. Reaction conditions and results are shown in Table 3.

TABLE 3

| Entry | Solvent | T (° C.) | t (hrs) | Conv (%) | Yield (%) |
|---|---|---|---|---|---|
| 1 | Toluene | 70 | 24 | 100 | 70 |
| 2 | THF | 50 | | 90 | 45 |
| 3 | Dioxane | 70 | | 90 | 55 |
| 4 | Acetonitrile | 60 | | 20 | 0 |
| 5 | Ethanol | 60 | | 80 | 50 |
| 6 | H₂O | 70 | | 10 | 0 |

### Example 4: Cyclization of Methallyl Indomuscone to Aromatic Cyclized Compound with Various Heterogeneous Acid Catalyst

In a 8 ml vial equipped with a magnetic stir bar and containing certain heterogeneous acid catalyst (1.04 g), a solution of Methallyl Indomuscone (1.04 g, 4 mmol) in toluene (2 ml) was added. The reaction mixture was open to the air and stirred at 70° C. for 24 hrs. After that time, an aliquot was dissolved in AcOEt (1 ml) and filtered through a 20 μm nylon filter, and the resulting filtrate solution was analyzed by GC and GC-MS. Reaction conditions and results are shown in Table 4.

TABLE 4

| Entry | Acid Catalyst | Conv (%) | Yield (%) |
|---|---|---|---|
| 1 | Zeolite H-Beta (CP-811) | 55 | 25 |
| 2 | Zeolite H-USY (CBV-720) | 40 | 10 |
| 3 | Zeolite H-USY (CBV-760) | 40 | 10 |
| 4 | Zeolite H-USY (CBV-780) | 30 | 10 |
| 5 | Zeolite H-USY (CBV-400) | 50 | 15 |
| 6 | Sulfonic Acid Resin Amberlyst 15 | 40 | 5 |
| 7 | Sulfonic Acid Resin Amberlyst 16 | 45 | 5 |
| 8 | Sulfonic Acid Resin Amberlyst 20 | 45 | 5 |
| 9 | Zeolite NaX | 0 | 0 |
| 10 | Zeolite NaY | | |

Sodium zeolites NaX and NaY are neutral (no Brønsted acid sites) and did not generate the Aromatic Cyclized Compound.

### Example 5: Cyclization of Methallyl Indomuscone to Aromatic Cyclized Compound with pTsOH at 70° C. For Various Reaction Time

In a 8 ml vial equipped with a magnetic stir bar and containing the acid catalyst pTsOH (140 mg), a solution of Methallyl Indomuscone (1.04 g, 4 mmol) in toluene (2 ml) was added. The reaction mixture was open to the air and stirred at 70° C. for a certain reaction time. After that time, the mixture was cooled and neutralized with 10% aqueous sodium hydrogen carbonate solution. The aqueous phase was extracted with hexane and washed with brine. The combined organic phases were dried over MgSO₄, filtered and concentrated under vacuum. An aliquot was dissolved in AcOEt (1 ml) and filtered through a 20 μm nylon filter, and the resulting filtrate solution was analyzed by GC and GC-MS. Reaction conditions and results are shown in Table 5.

TABLE 5

| Entry | Reaction Time (hrs) | Yield (%) |
|---|---|---|
| 1 | 0.25 | <1 |
| 2 | 0.5 | 2 |
| 3 | 1 | 7 |
| 4 | 2 | 11 |
| 5 | 4.5 | 24 |
| 6 | 6.5 | 33 |
| 7 | 8 | 38 |
| 8 | 11.5 | 48 |
| 9 | 13.5 | 51 |
| 10 | 24 | 70 |

### Example 6: Alkylation of Indomuscone with Methallyl Chloride to Make Methallyl Indomuscone

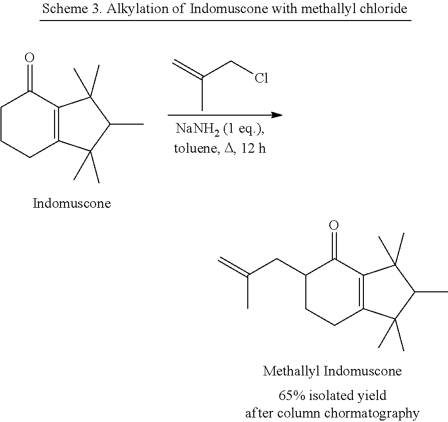

A suspension of sodium amide (242 mg, 5.6 mmol) in toluene (1.8 ml) was slowly added during 5-10 min (minutes) to a 3 M toluene solution of Indomuscone (1 ml, 5.6 mmol) in a 50 ml round-bottom flask equipped with a magnetic stir bar at room temperature under continuous stirring. After 20 min, a 3 M toluene solution of methallyl chloride (550 μl, 5.6 mmol) was added into the flask, and the stirring continued for 12 hrs while the reaction mixture was refluxed. After cooling, the mixture was neutralized with HCl aqueous solution, extracted with ether and washed with brine. The combined organic phases were dried over MgSO₄, filtered and concentrated under vacuum. Flash column chromatography (2% AcOEt in hexane) gave 946 mg (65% yield) of Methallyl Indomuscone as a yellow oil.

### Example 7a: Alkylation of Indomuscone with Isobutylene Oxide to Make Hydroxyisobutyl Indomuscone

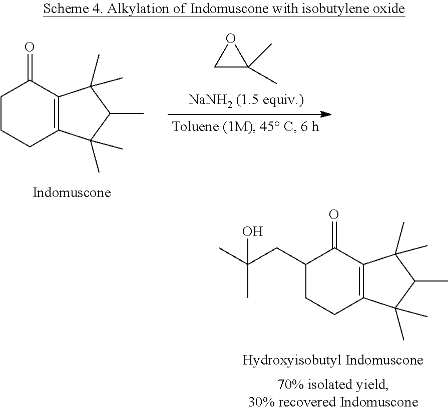

Indomuscone (10 ml, 45 mmol) was slowly added during 15 min to a suspension of sodium amide (2.5 g, 66 mmol) in toluene (45 ml) in a 100 ml round-bottom flask equipped with a magnetic stir bar at room temperature under continuous stirring (750 rpm) and under N₂ atmosphere. Then the mixture was heated to 45° C. After 30 min at 45° C., isobutylene oxide (3.5 ml, 66 mmol) was slowly added into the flask and the stirring continued for 6 hours. After cooling, the reaction mixture was neutralized with NH₄Cl. The aqueous phase was extracted with ethyl acetate and washed with brine. The combined organic phases were dried over MgSO₄, filtered and concentrated under vacuum. Flash chromatography (1% AcOEt in hexane) gave 8.8 g (70% yield) of Hydroxyisobutyl Indomuscone as a yellow oil.

### Example 7b: Alkylation of Indomuscone with Isobutylene Oxide to Make Hydroxyisobutyl Indomuscone

Same process as Example 7a was conducted in Example 7b except that the reaction temperature was 60° C. and the reaction time was 12 hours. The yield of Hydroxyisobutyl Indomuscone was 80%.

### Example 8: Hydrogenation of Aromatic Cyclized Compound to Make Musk Indenofuran

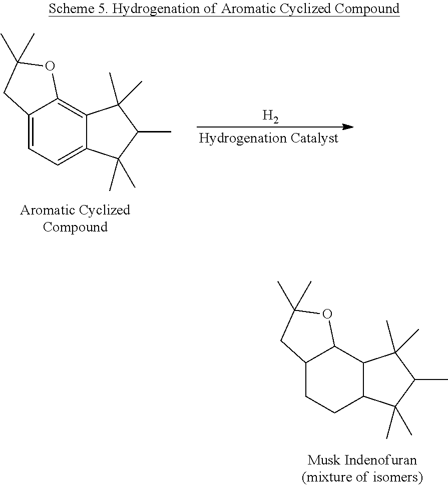

The hydrogenation catalyst used in Example 8 was rhodium loaded on carbon support (Rh/C). The content of the rhodium element was 5 wt % based on the total weight of the catalyst. The solvent used in Example 8 was ethanol or THF. The hydrogenation catalyst (Rh/C 5 wt %, 145 mg), solvent (ethanol or THF, 1 ml) and Aromatic Cyclized Compound (50 mg, 0.2 mmol) were charged into a 4 ml vial containing a magnetic stir bar. Then, the reaction vial was capped with a septum equipped with a needle and set in an alloy plate, which was then placed into a 300 ml autoclave. Once sealed, the autoclave was purged three times with 20 bars of hydrogen, then pressurized to 40 bars of hydrogen and placed into an aluminium block, which was pre-heated at 140° C. After 18 hours, the autoclave was cooled in an ice bath, and the remaining hydrogen gas was carefully released. Finally, the reaction mixture containing Musk Indenofuran was diluted with ethyl acetate and analysed by GC and GC-MS. Reaction conditions and results are shown in Table 6.

TABLE 6

| Entry | Solvent | T (° C.) | t (hrs) | Conv (%) | Yield (%) |
|---|---|---|---|---|---|
| 1 | Ethanol | 140 | 18 | 90 | 90 |
| 2 | THF | | | 60 | 60 |

### Example 9: One-Pot Cyclization/Hydrogenation of Hydroxyisobutyl Indomuscone to Make Musk Indenofuran

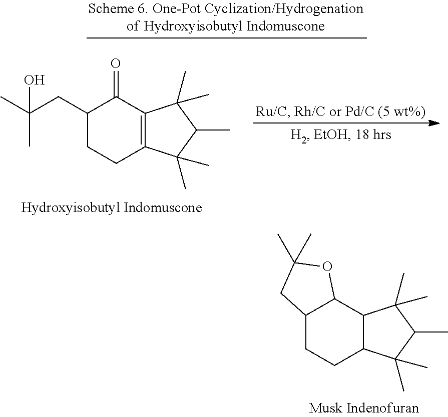

The hydrogenation catalyst used in Example 9 was ruthenium, rhodium, or palladium loaded on carbon support (Ru/C, Rh/C, or Pd/C). The content of the ruthenium, rhodium, or palladium element was 5 wt % based on the total weight of the corresponding catalyst. The solvent used in Example 9 was ethanol. The hydrogenation catalyst, Hydroxyisobutyl Indomuscone (125 mg, 0.7 mmol) and ethanol were charged into a 4 ml reaction vial containing a stirring bar. Then the reaction vial was capped with a septum equipped with a needle and set in an alloy plate, which was then placed into a 300 ml autoclave. Once sealed, the autoclave was purged three times with hydrogen, then pressurized to 30 bars or 50 bars and placed into an aluminium block, which was pre-heated at 130° C. or 150° C. After 18 hours, the autoclave was cooled in an ice bath, and the remaining hydrogen gas was carefully released. Then an aliquot of the product mixture containing Musk Indenofuran was dissolved in ethyl acetate (1 ml), the mixture was filtered through a 20 mm nylon filter and the resulting solution was analyzed by GC and GC-MS. Reaction conditions and results are shown in Table 7.

TABLE 7

| | Cat | H₂ | T | Solvent | Conv | Yield |
|---|---|---|---|---|---|---|
| Entry | (mol %) | P | (° C.) | (ml) | (%) | (%) |
| 1 | Rh/C (5) | 30 | 130 | ethanol (0.1) | 100 | 70 |
| 2 | Pd/C (5) | 30 | | | | 60 |
| 3 | Ru/C (5) | 30 | | | | 67 |
| 4 | Ru/C (1) | 30 | | | 95 | 55 |
| 5 | | | | | 90 | 35 |
| 6 | Ru/C (0.1) | 30 | | ethanol (0.4) | 50 | 16 |
| 7 | | | | | 40 | 10 |
| 8 | Ru/C (1) | 50 | 150 | ethanol (0.4) | 100 | 63 |
| 9 | | | | | | 40 |
| 10 | Ru/C (0.5) | 50 | | ethanol (0.4) | | 62 |
| 11 | | | | | | 42 |
| 12 | Ru/C (0.1) | 50 | | ethanol (0.4) | 60 | 23 |
| 13 | | | | | 40 | 15 |

Note:
“H₂ P” means hydrogen pressure in bars; “Cat (mol %)” means the catalyst and its Rh, Pd, or Ru amount in mole percentage based on the molar amount of the Hydroxyisobutyl Indomuscone starting material.

### Example 10: Bromination of Aromatic Cyclized Compound to Make Aromatic Bromide Compound

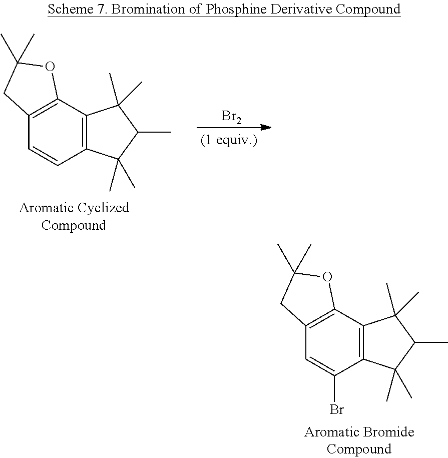

Aromatic Cyclized Compound (1.95 g, 7.5 mmol) was placed in a 250 ml round-bottom flask equipped with a magnetic stirrer. Dichloromethane (50 ml) was added into the flask. After the solid was dissolved at room temperature under magnetic stirring, Br₂ (390 μl, 7.5 mmol) was added dropwise into the flask. After the addition of Br₂ was completed, the resulting reaction mixture was further stirred for 15 minutes, and then treated with Na₂S₂O₃ aqueous solution (aq.), NaHCO₃ (aq.) and brine. The organic phase was dried over Na₂SO₄ and filtered. Volatiles were removed from the filtrate under vacuum to give the Aromatic Bromide Compound as a yellow oil (2.1 g, 87% yield).

### Example 11: Phosphination of Aromatic Bromide Compound to Make Phosphine Derivative Compound

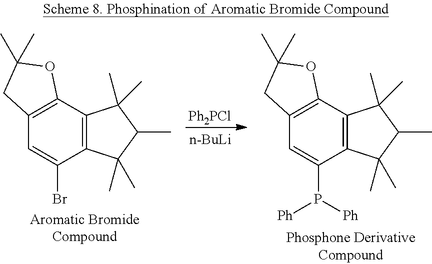

The Aromatic Bromide Compound (340 mg, 1 mmol) was placed in a dried 10 ml round-bottom flask equipped with a magnetic stirrer, dissolved in anhydrous THF (2 ml) under nitrogen atmosphere, and cooled to −78° C. Then, n-BuLi 2.5 M in hexane (0.5 ml, 1.25 mmol) was added dropwise into the flask, and the resulting reaction mixture turned from yellow to orange color. The reaction mixture was magnetically stirred at −78° C. for additional 15 min before chlorodiphenylphosphine (Ph₂PCl, 180 μl, 1.0 mmol, 1 eq.) was added into the flask at once. The reaction mixture was then warmed to room temperature for 90 min under stirring. The reaction mixture was then quenched with NH₄Cl (aq.) and washed with water and brine consecutively. The organic phase was dried over Na₂SO₄ and filtered. The desired phosphine derivative compound product ((2,2,6,6,7,8,8-heptamethyl-3,6,7,8-tetrahydro-2H-indeno[4,5-b]furan-5-yl) diphenyl phosphane) was purified by column chromatography and then by preparative thin-layer chromatography [TLC, 3% AcOEt in n-hexane; Rt (10% AcOEt in n-hexane)=0.65] to give the desired phosphine derivative compound (colorless solid, 150 mg, 34% yield) after removing volatiles under vacuum.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination.

1. A process comprising: cyclizing a starting material in the presence of oxygen, an acid catalyst and a solvent in a reaction zone to form 2,2,6,6,7,8,8-heptamethyl-3,6,7,8-tetrahydro-2H-indeno[4,5-b]furan (Aromatic Cyclized Compound), wherein the acid catalyst is a homogeneous acid catalyst with pKa of no more than zero and/or a heterogeneous acid catalyst, and the starting material is 1,1,2,3,3-pentamethyl-5-(2-methylallyl)-1,2,3,5,6,7-hexahydro-4H-inden-4-one (Methallyl Indomuscone) and/or 5-(2-hydroxy-2-methylpropyl)-1,1,2,3,3-pentamethyl-1,2,3,5,6,7-hexahydro-4H-inden-4-one (Hydroxyisobutyl Indomuscone).

2. The process of claim 1, wherein the starting material is Methallyl Indomuscone.

3. The process as in claim 1, wherein the acid catalyst is the homogeneous acid catalyst.

4. The process of claim 3, wherein the homogeneous acid catalyst is selected from the group consisting of p-toluenesulfonic acid, methanesulfonic acid, sulfuric acid, triflic acid, trifluoromethanesulfonimide, trifluoromethanesulfonamide, and mixtures thereof.

5. The process of claim 4, wherein the homogeneous acid catalyst is selected from the group consisting of p-toluenesulfonic acid, methanesulfonic acid, and mixtures thereof.

6. The process as in claim 1, wherein the acid catalyst is the heterogeneous acid catalyst.

7. The process of claim 6, wherein the heterogeneous acid catalyst is selected from the group consisting of alumina, silica-alumina, zeolite, silico-alumino-phosphate, aluminophosphate, sulfated zirconia, zirconia, sulfonic acid resin, and mixtures thereof.

8. The process of claim 7, wherein the heterogeneous acid catalyst is selected from the group consisting of zeolite, sulfonic acid resin, and mixtures thereof.

9. The process of claim 8, wherein the heterogeneous acid catalyst is zeolite.

10. A process comprising: transforming a starting material in the presence of an acid catalyst and a solvent in a reaction zone into an intermediate compound selected from the group consisting of 1,1,2,3,3-pentamethyl-5-(2-methylallyl)-2,3-dihydro-1H-inden-4-ol (Methallyl Phenol Compound), 2,2,6,6,7,8,8-heptamethyl-3,4,5,6,7,8-hexahydro-2H-indeno[4,5-b]furan (Endocyclic Diene I), 2,2,6,6,7,8,8-heptamethyl-3,3a,4,6,7,8-hexahydro-2H-indeno[4,5-b]furan (Endocyclic Diene II), and mixtures thereof, wherein the transforming process is conducted under substantial absence of oxygen, the acid catalyst is a homogeneous acid catalyst with pKa of no more than zero and/or a heterogeneous acid catalyst, and the starting material is 1,1,2,3,3-pentamethyl-5-(2-methylallyl)-1,2,3,5,6,7-hexahydro-4H-inden-4-one (Methallyl Indomuscone) and/or 5-(2-hydroxy-2-methylpropyl)-1,1,2,3,3-pentamethyl-1,2,3,5,6,7-hexahydro-4H-inden-4-one (Hydroxyisobutyl Indomuscone).

11. The process of claim 10, wherein the starting material is Methallyl Indomuscone.

12. The process as in claim 10, wherein the acid catalyst is the heterogeneous acid catalyst.

13. The process as in claim 10 further comprising transforming the intermediate compound in the presence of oxygen into 2,2,6,6,7,8,8-heptamethyl-3,6,7,8-tetrahydro-2H-indeno[4,5-b]furan (Aromatic Cyclized Compound).

14. The process as in claim 10, wherein the intermediate compound comprises an endocyclic diene compound selected from the group consisting of Endocyclic Diene I, Endocyclic Diene II, and mixtures thereof, and the endocyclic diene compound is hydrogenated to form 2,2,6,6,7,8,8-heptamethyldecahydro-2H-indeno[4,5-b]furan (Musk Indenofuran).

15. A process comprising:
(a) alkylating Indomuscone with methallyl chloride under conditions effective to produce Methallyl Indomuscone,
(b) cyclizing Methallyl Indomuscone under conditions effective to produce Aromatic Cyclized Compound, and
(c) hydrogenating Aromatic Cyclized Compound under conditions effective to produce Musk Indenofuran.

16. (canceled)

17. (canceled)

18. (canceled)

19. (canceled)

20. (canceled)
